(19) United States
(12) Patent Application Publication
(10) Pub. No.: US 2009/0018516 A1
(43) Pub. Date: Jan. 15, 2009

(54) Biodegradable and disposable feminine hygiene product

(76) Inventor: Shala Kelly, Neptune, NJ (US)

Correspondence Address: Shala Kelly, 527 Myrtle Avenue, Neptune, NJ, 07753, US

(21) Appl. No.: 12/214,780

(22) Filed: Jun. 23, 2008

Related U.S. Application Data
(60) Provisional application No. 60/937,250, filed on Jun. 25, 2007.

(51) Int. Cl. A61F 13/15

(52) U.S. Cl. 604/364

(57) ABSTRACT

A water-degradable and biodegradable, environmentally-friendly feminine hygiene product that can be utilized and then disposed of by flushing the entire product without disassembly or separation of parts.

### CROSS REFERENCE TO RELATED APPLICATIONS

This application claims the benefit of an earlier filing date under 35 USC 119(e) for provisional application No. 60/937,250, with a filing date of Jun. 25, 2007.

### BACKGROUND OF THE INVENTION

1. Field Of The Invention

The present invention relates to a biodegradable, water-degradable or flushable hygiene product. More particularly, this invention relates to a feminine hygiene product containing at least a biodegradable or water-degradable absorbent layer, and a water-degradable structural layer.

2. Background of the Invention

Personal hygiene products, such as infant diapers, sanitary napkins, and adult incontinence garments are generally constructed from a number of varying components and materials. Such articles typically have, in some configuration, a structural layer, an absorbent later, and a barrier or baffle constructed of a liquid repellent film material. This repellent material is appropriately constructed to minimize or prevent the release of the absorbed liquid from the article and to obtain greater utilization of the absorbent capacity of the product. Both the backing layers and barrier layers of these products tend to be non-biodegradable, and non-water-degradable. As a result, these portions of the product lead the entire product to be non-flushable, or these portions must be removed from the product prior to flushing. Neither instance is preferred, as both lead to landfill waste and consume unnecessary time and effort in the separation, preparation and disposal.

Although such products are relatively inexpensive, sanitary and easy to use, disposal of a used product is not without its problems. With greater interest being placed in protecting the environment, there is a need to develop materials that are more compatible with the existing and developing waste disposal technologies while still delivering the performance consumers have come to expect. An ideal disposal alternative would be to use municipal sewage treatment and private residential septic systems. Products suited for disposal in sewage systems can be flushed down a convenient toilet or related device connected to a septic tank or septic system are termed “flushable.”

While flushing such articles would be convenient, the liquid repellent material and/or backing materials normally do not disintegrate in water, and tend to plug toilets and sewer pipes. The resulting damage from damaged toilets and water pipes in residential homes and commercial complexes results in staggering financial losses. To prevent such damage, it therefore becomes necessary for the user to separate the barrier film material from the absorbent article prior to flushing, which in turn creates additional landfill and superfluous environmental damage.

Desirably, a commercial, water degradable or flushable product should be relatively responsive to water and be transportable in a sewer system. Commercially available water-soluble polymers may include, but are not limited to poly(ethylene oxide) (PEO), poly(vinyl alcohol) (PVOH), acrylamide polymers, acrylic acid-based polymers, and cellulose derivatives, possess the desired characteristics for flushability, such as water solubility and/or water dispersibility. The requirements for a functional and flushable product provide a substantial challenge in finding suitable materials with the desired properties. In an attempt to overcome the flushability problem of a water resistant film several pieces of prior art have modified the water resistant polymer in some capacity. However, to date, no prior art has modified the feminine hygiene product in a way that is both effective, protective and flushable.

It is therefore an object of the invention to provide a flushable, biodegradable and/or water-degradable hygiene product which consists at least of a flushable biodegradable absorbent layer, a water-degradable structural layer, and an attachment layer.

### DESCRIPTION OF DRAWINGS

FIG. 1. shows a lateral view of one embodiment of the invention;

FIG. 2 shows a lateral view of one embodiment of the invention; and

FIG. 3 shows an exploded lateral view of another embodiment of the invention, including flaps.

### DETAILED DESCRIPTION OF THE INVENTION

As used herein, “water degradable” means a protein-based film, a polymer film, or other such chemically-synthesized product which can be dispersed or molded into a film or layer, having some range of pliability or flexibility, when immersed in water for 1-60 seconds, will have been modified, in an entropic state, to enter solution with the surrounding water molecules.

The term “feminine hygiene product” or “personal care product” means articles such as infant diapers, sanitary napkins and/or tampons, adult incontinence garments, and the like.

By the term “structural layer” or “barrier layer” is meant that component of an infant diaper, sanitary napkins and/or tampons, adult incontinence garment or the like which is worn during normal use furthest (or most distal) from the user's body and which serves to minimize or prevent the exudation of the absorbed liquid. This layer can serve strictly to provide mechanical support to the absorbent layer, or it can serve both as mechanical support, and as a physical barrier between the absorbent layer and the user's clothing.

The term “absorbent layer” is meant to represent either a fiber or polymeric layer of a personal care product, held closest to the user's body, which when contacted by fluids, will retain said fluids under normal physiological conditions. The absorbent layer may be comprised of a singular layer, multiple layers, or a matrix formation of components.

The term “attaching layer” is mean to represent either a chemical or physical device which can temporarily, but rigorously, adhere the invention to the undergarments, outer-garments or physical person of the user. Such chemical or physical device can be adhesive tapes, adhesive gels, or similarly adhesive materials, or it can be Velcro®, or similarly designed hook-and-loop structures, or other adhesives which the person skilled in the art can reasonably anticipate might attach the present invention to clothing or an individual.

The term “barrier layer” means a water-resistant layer which can either be attached to the attaching layer or to the structural layer. The barrier layer is re-usable, and can be attached, detached and re-attached to another product. The barrier layer is washable and generally manufactured of sterile or bacteria-resistant materials.

Although the present invention is described with reference to a water-degradable polymer and absorbent layer and, in particular, to personal care articles having a structural layer, and absorbent layer and comprising such water-degradable and/or bio-degradable films, one skilled in the art will understand that the composition of the invention can be modified to include adhesive devices, liner devices, liner flaps and all other feminine personal hygiene structures that may be appropriate to maximize both the biodegradability, absorbency and ease of use of the product.

The present invention consists of a small, thin, comfortably shaped, absorbent layer having some combination of at least two of the following: a body-contacting surface, absorptive fibers, positioning means, and in some embodiments, a barrier layer which is also bio-degradable. The invention can be securely yet removably attached to an undergarment, or directly to the user via the attachment layer.

The body-contacting surface may be a porous, substantially planar flexible polymeric coating on the surface of the absorbent layer in an integral pad construction. Such a surface, due to the flatness of the polymeric coating, creates a comfortable contact with the user's person.

The absorptive layer of the invention is comprised of a bio-degradable and/or water-soluble fiber. In one embodiment of the present invention, this layer may comprise a spatially controlled blend of (1) absorbent cellulose paper fluff and (2) a hydrophobic textile fiber distributed in a web. In another embodiment, the absorptive layer may be comprised of absorbent treated non-woven fibers. In other embodiments of the present invention, the absorbent layer can utilize a variety of water immobilizing materials to increase fluid capacity or minimize bulk. Such embodiments may contain one or more of the following components, but shall not be limited to: cotton or similar textiles, polysaccharides, non-cross-linking polymers, modified polymers, propylene-based co-polymers, non-propylene based co-polymers. It will, of course, be appreciated that the absorptive portion of the invention may be either the top portion, the central portion, or one of many portions in an integrally formed pad member having a porous, planar, flexible polymeric coating as its top surface.

The structural layer, or binding layer, may be substantially planar, or may be molded, formed or otherwise conformationally adhered to the invention, so as to provide the absorbent layer with structural integrity. The structural layer may also potentially serve as a final barrier structure to excess bodily fluids which may exude from the absorbent layer. In one embodiment, the structural layer is comprised of at least one water-soluble-polymer, which is sensitive to the general pH and ionic requirements of dissolution in water. In this instance, the structural layer will not dissolve simply upon contact with the majority of bodily fluids, which contain high levels of solutes, and vary in the acidic or basic levels of pH. In this case, the structural layer also serves as a barrier layer, preventing bodily fluids from being exuded from the absorbent layer and potentially permeating the user's clothing. In another embodiment, the structural layer is comprised of a slightly more rigid polymer, which is conformationally adhered to the absorbent layer. In this embodiment, the structural layer provides mechanical support to the fibers and polymers, lending a planar shape, or a shape that is otherwise regarded as conforming to the body of the user.

The adhesive layer can be attached by additional adhesive means, or can be intergrally molded into the structural layer or the absorbent layer, depending on the conformation of the invention. The adhesive layer is expected to hold the invention rigorously in place, to avoid disconnection of the invention to the user, and to maximize the surface area contact of one or more of the layers to the user. However, upon the determination by the user that the specific invention product has maximized its absorbency and should be flushed, the adhesive layer should yield cleanly to no more than the pulling force of two to three fingers of the average human hand.

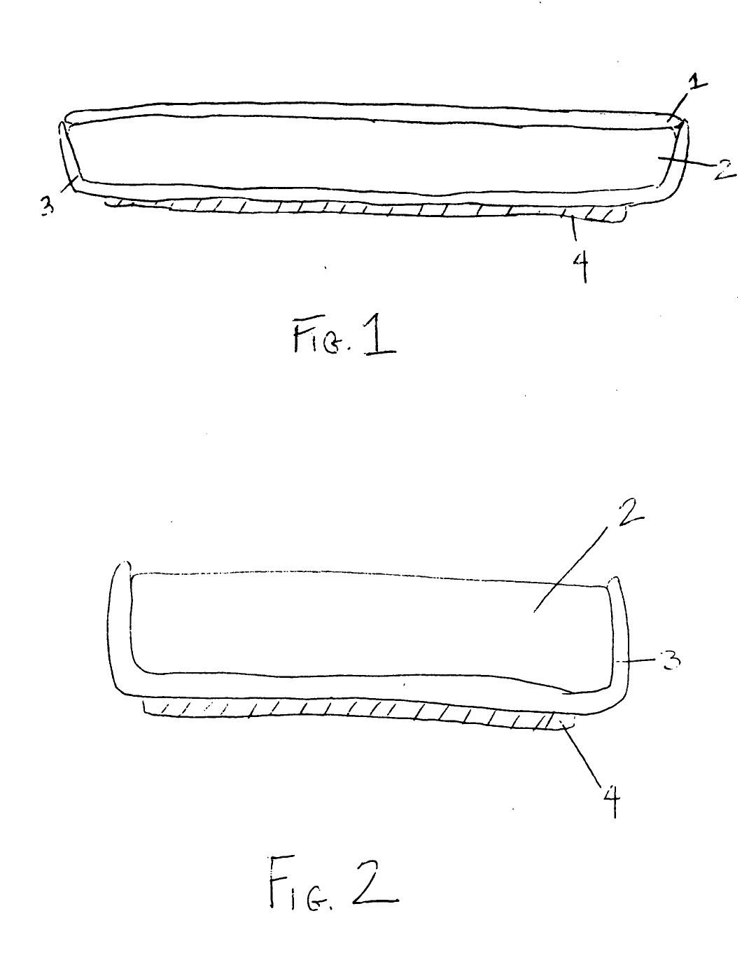

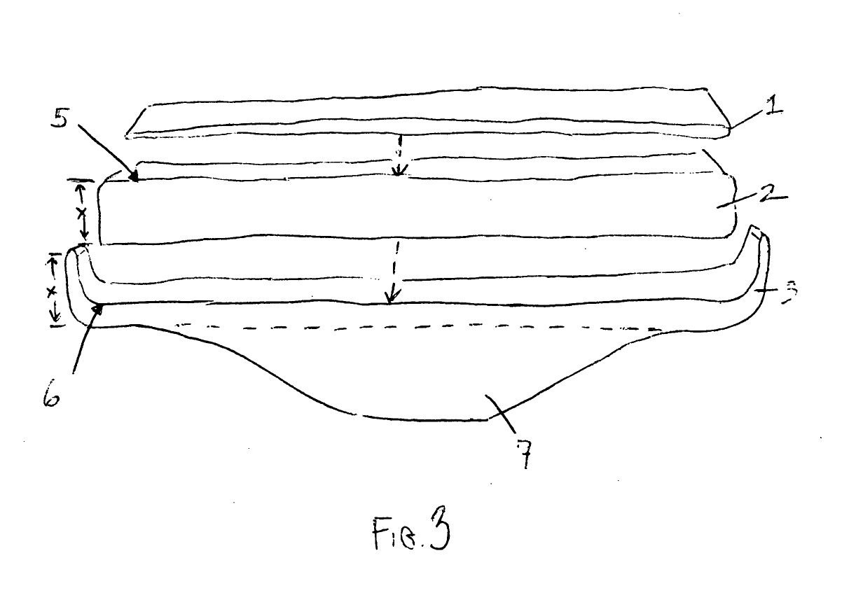

In one embodiment, as shown-in FIG. 1 the body-contacting surface coating (1) is adhered directly to the absorbent layer (2). Said absorbent layer is adhered directly to the structural layer (3) in a substantially planar formation. This embodiment is flexible, and will conform to the contours of the user, so as to provide maximum surface-area contact. On the distal surface of the structural layer is located an attaching layer, such as an adhesive tape or gel. This attaching layer (4) allows the user to bond the invention with their undergarments, or directly to their outer layer of clothing. This bonding of the invention to the undergarments, etc . . . places the invention in the position the user feels most appropriate. As bodily fluids, such as menses, exit the user's body, said fluids will come into immediate contact with the absorbent layer, and shall be held within the matrices of said absorbent layer. A barrier layer can be affixed to the attachment layer, or to the structural layer.

When the user determines the individual invention product has been maximized, the user may disconnect the invention from their person, or their clothing, and place the invention, in it's entirety—that is, without having to remove, separate, detach, or otherwise modify the invention—in a toilet, water closet, or similarly related plumbing device that will allow matter to flow freely into a septic receptacle. This action, for the purposes of this invention, is considered “flushing” the invention. Upon contact with the water in the toilet and/or septic receptacle, the more rigid structural layer will become significantly or entirely dissolved over a short period of time. It is expected that the initial dissolution in water of the structural layer will occur within 1 to 60 seconds. Significant dissolution may occur in the water for an extended time period after flushing. The absorbent layer is initially flushable and will either quickly biodegrade or become dissolved.

It is a consideration that the invention, in other embodiments, such as those shown in FIG. 3 will contain at least a top proximal layer of body-contacting surface coating (1), adhered distally to the proximal region (5) of the absorbent layer (2), adhered distally to the proximal region (6) of the structural layer (3). On either side of the structural layer are biodegradable flaps (7) which extend laterally in a tapered fashion. Such flaps can be extended around the undergarment of the user, to provide additional protection from any fluids which might leak from the absorbent layer. These flaps are comprised of the same materials as the structural layer, and may or may not be integrally formed with said structural layer. While not shown, as in the previous embodiment the attaching layer (4), is attached to the distal region of the structural layer (3).

1. A water-degradable feminine hygiene product which can be disposed of by flushing said product in its entirety;

2. A feminine hygiene product according to claim 1 wherein said feminine product comprises a water-degradable absorbent layer, and a water-degradable structural layer;

3. A feminine hygiene product according to claim 2, wherein said feminine product also comprises of an attaching layer

3. A feminine hygiene product according to claim 2, wherein said water-degradable absorbent layer will enter an aqueous solution in 1-24 hours of being immersed in water;

4. A feminine hygiene product according to claim 2, wherein said water-degradable absorbent layer will enter an aqueous solution in 10 minutes to 1 hour of being immersed in water;

5. A feminine hygiene product according to claim 2, wherein said water-degradable absorbent layer will enter an aqueous solution within 1-10 minutes of being immersed in water;

6. A feminine hygiene product according to claim 2, wherein said water-degradable absorbent layer will enter an aqueous solution within 1 second to 60 seconds of being immersed in water;

7. A bio-degradable feminine hygiene product which can be disposed of in an environmentally-friendly fashion, by decomposing rapidly in aqueous solution
